SEQUENCE LISTING (4 sequences)

SEQ ID NO: 1 - DNA, 24 bp, Artificial Sequence
  Human actin primer - HBAC1650
tcccccaacttgagatgtatgaag

SEQ ID NO: 2 - DNA, 25 bp, Artificial Sequence
  Human actin primer - HBAC1717
aactggtctcaagtcagtgtacagg

SEQ ID NO: 3 - DNA, 20 bp, Artificial Sequence
  Human actin primer - HBAC 1355
atcccccaaagttcacaatg

SEQ ID NO: 4 - DNA, 20 bp, Artificial Sequence
  Human actin primer - HBAC1472
gtggcttttaggatggcaag

(19) United States
(12) Patent Application Publication
(10) Pub. No.: US 2006/0063170 A1
(43) Pub. Date: Mar. 23, 2006

(54) Determination of RNA quality

(75) Inventors: Mark G. Erlander, Redwood City, CA (US); Li Ding, (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arcturus Bioscience, Inc., Mountain View, CA (US)

(21) Appl. No.: 11/084,582

(22) Filed: Mar. 18, 2005

Related U.S. Application Data
(60) Provisional application No. 60/554,527, filed on Mar. 18, 2004.

(51) Int. Cl. C12Q 1/68; C12P 19/34

(52) U.S. Cl. 435/6; 435/91.2

(57) ABSTRACT

This invention relates to the determination of RNA quality, or RNA integrity, in a biological sample. The extent of RNA degradation, or retention of RNA integrity, is determined based upon the comparison of the relative amount of two sequences of a representative RNA molecule in the sample. Compositions and methods related to the determination are provided to assess RNA quality.

### RELATED APPLICATIONS

This application claims benefit of priority from Provisional U.S. Patent Application 60/554,527, filed Mar. 18, 2004, which is hereby incorporated in its entirety as if fully set forth.

### FIELD OF THE INVENTION

This invention relates to the determination of RNA quality, or RNA integrity, in a biological sample. Compositions and methods related to the determination are provided to assess RNA quality.

### BACKGROUND OF THE INVENTION

The detection of gene expression has become an increasingly important area in the biological sciences, including applications with clinical relevance. Such applications include the interest in retrospective studies that correlate the expression of one or more gene sequences in a patient sample with information from subsequent clinical follow up. Other applications include the use of patient samples for analysis by microarrays for prospective clinical trials. Yet additional applications include diagnostic tests based upon the expression of one or more gene sequences in a patient sample.

Detecting the expression of particular sequences as MRNA is a format used for many of these gene expression based methods. But there are many factors which can affect the quality and quantity of the MRNA, as well as RNA in general, in a sample. For example, RNA degrades with time. As such, the RNA in various biological samples can vary significantly in both quantity and quality.

Additionally, many patient samples are fixed in 10% neutral buffered formalin followed by paraffin embedding to result in formalin fixed and paraffin embedded (FFPE) samples. FFPE samples are favored because they preserve cells and tissue morphology much like in fresh tissue and allows for subsequent processing while also preventing bacterial degradation without the need for freezing.

The process of obtaining FFPE samples, however, can add to the level of RNA degradation while also introducing a number of variables in the extent of degradation. Such variables include the time from excision of a biological sample (or biopsy) from a patient to fixation in formalin; the size of the biological sample and thus the rate of fixation; and the amount used for fixation in formalin. Additionally, the formalin fixation process introduces RNA fragmentation and base modification by addition of mono-methylol (—CH₂OH) groups. The quality and quantity of RNA in a sample can significantly affect the ability to use the RNA in applications as described above.

Previous methods for the determination of RNA quality include simple gel electrophoresis and the use of a Bioanalyzer™ made by Agilent Technologies. While these methods have been fairly reliable for RNA from fresh or frozen samples, they have been of limited usefulness for FFPE samples.

Citation of documents herein is not intended as an admission that any is pertinent prior art. All statements as to the date or representation as to the contents of documents is based on the information available to the applicant and does not constitute any admission as to the correctness of the dates or contents of the documents.

### SUMMARY OF THE INVENTION

This invention provides compositions and methods for their use in the determination, or assessment, of RNA quality and/or quantity. The concept of RNA quality, or integrity, includes at least the degree of RNA degradation, or conversely the degree of RNA intactness, in general within a biological sample. The invention is based upon the comparison of the relative amount of two sequences of a representative RNA molecule in the sample. The compositions of the invention include those containing the necessary reagents for the determination of RNA quality and/or quantity as well as those containing the products used to make the determination.

The invention provides for the assessment of RNA degradation or fragmentation in a sample by reference to the condition of polyadenylated RNAs in the sample. This is conveniently and advantageously conducted by the conversion of polyadenylated RNAs to the corresponding cDNAs by use of reverse transcription with an oligo dT containing primer or other analogous method. In alternative embodiments, the assessment can be made by use of non-polyadenylated RNA or cDNAs prepared via reverse transcription using random primers or specific (non-oligo dT) primers.

Thus the invention provides reagents necessary for use in a method of assessing the quality of RNA in a biological sample by first obtaining RNA from said sample. In some embodiments, the RNA includes at least one expressed polyadenylated RNA transcript to be used for the determination of RNA quality. The RNA preferably also includes at least one MRNA species of interest. The biological sample preferably contains at least one cell from the subject from whom the sample was obtained. The subject is preferably a human being, optionally afflicted with, or suspected of being afflicted with, a disease or other unwanted condition, such as, but not limited to, cancer. The sample may be an FFPE sample, a fresh sample, or a frozen sample.

Alternatively, the invention may be practiced in situ within a biological sample, or a portion thereof, whereby the expressed RNA found in said sample (or portion thereof) is used without isolation.

In some embodiments, the invention utilizes quantitative determinations of the levels of expression of different sequences within a single polyadenylated RNA transcript among the expressed RNA in a biological sample to determine RNA quality. The polyadenylated RNA molecules in the sample are used to prepare cDNA molecules by reverse transcription using an oligo dT primer or analogous primer, such as, but not limited to, a poly-dT primer or a specific primer complementary to a 3′ portion of an RNA molecule. Thus the extent of fragmentation/degradation of the polyadenylated RNA population is reflected in the prepared cDNA population.

Different sequences of any one of the resultant cDNA molecules may be used in the practice of the invention. The different sequences of a cDNA molecule used in the practice of the invention are those near the 3′ end and those further from the 3′ end, both defined in relation to the corresponding polyadenylated RNA molecule.

In some embodiments, the invention thus provides for the determination of the amount of a first sequence quantitatively amplified from a cDNA molecule where the first sequence is 5′ to the nucleotide corresponding to the start of the polyadenylate (polyA) tail of the polyadenylated RNA from which the cDNA was reverse transcribed. Alternatively, the first sequence may contain the nucleotide corresponding to the nucleotide of the polyadenylated RNA before the start of the polyA tail.

In other embodiments, the invention provides for the assessment of the RNA quality, or the degree of RNA degradation, in a sample from a biological source by comparing the amounts of two sequences of a representative RNA molecule in the sample. The two sequences may be quantitatively amplified from the RNA molecule to facilitate their detection and the assessment of RNA integrity. The amplification may be of a cDNA molecule prepared by reverse transcription of the RNA molecule. The reverse transcription may be of many or all RNA molecules in the sample. Non-limiting means to conduct reverse transcription include use of an oligo-dT or poly-dT primer; use of random primers to synthesize the first cDNA strand; or use of one or more specific primers to synthesize the first cDNA strand. If a dT primer is used to prepare cDNA from polyadenylated RNA molecules, then the invention may be practiced by comparing sequences of a representative polyadenylated RNA molecule. If a specific primer is used to prepare only cDNA corresponding to one RNA molecule, then the invention may be practiced by assessing two sequences in that cDNA.

The locations of the two sequences in the RNA molecule, or the corresponding cDNA, are such that the first of the two is located 3′ relative to the location of the second sequence. Stated differently, the second of the two sequences is 5′ relative to the location of the first sequence. Thus the 5′ most nucleotide of the first sequence is closer to the 3′ end of the RNA molecule than the 5′ most nucleotide of the second sequence (and the 5′ most nucleotide of the second sequence is closer to the 5′ end of the RNA molecule than the 5′ end of the first sequence).

In some embodiments, the two sequences may be detected via their amplification, such as by PCR (polymerase chain reaction) to amplify each sequence as an amplicon. An amplicon contains the sequences of the primers used in the PCR reaction as well as the intervening sequence amplified by (and delineated by) the primers used. The invention also provides embodiments wherein the second sequence may comprise all or part of the first sequence. Stated differently, the second sequence may overlap in whole or in part with the first sequence. In alternative embodiments, the two sequences do not overlap.

In some embodiments of the invention, the first sequence (starting with the 5′ most nucleotide of the sequence) is within about 300 to about 500 nucleotides of the cDNA nucleotide corresponding to the start of the polyA tail in the polyadenylated RNA. In other embodiments, the first sequence may be more distant from the nucleotide(s) corresponding to the start of the polyA tail in a polyadenylated RNA.

The first sequence may be of any length, but shorter lengths that are suitable for quantitative amplification are preferred. Such lengths include those less than about 250 nucleotides, less than about 200 nucleotides, less than about 150 nucleotides, less than about 100 nucleotides, less than about 90 nucleotides, less than about 80 nucleotides, less than about 70 nucleotides, less than about 60 nucleotides, and less than about 50 nucleotides in length.

The first sequence may be detected by any suitable means, including, but not limited to, quantitative amplification. The quantitative amplification used may be any known in the art, with quantitative PCR (QPCR) using two primers capable of amplifying the first sequence being preferred. Non-limiting examples include the use of QPCR with molecular beacon (e.g. TaqMan®) probes or nucleic acid binding probes (e.g. SYBR Green). The two sequences may be simultaneously amplified and detected, such as by use of multiplex QPCR in one reaction, or be amplified and detected by separate reactions using material RNA obtained or cDNA derived from the same sample. Alternatively, the invention may be practiced by use of ligase chain reaction (LCR), preferably quantitative LCR (QLCR), to detect each of the two sequences described herein.

Through some embodiments, the invention provides a process that may be viewed as quantitative reverse transcription-PCR (QRT-PCR) given the reverse transcription process to produce cDNA molecules from RNA. While any suitable QPCR or QRT-PCR method may be used, methods comprising the use of labeled molecules or reporters (labels) that bind to allow detection of the amplified product are preferred.

The invention also provides embodiments for the determination of the amount of a second sequence from the same RNA (or corresponding cDNA molecule). This second sequence is preferably located 5′ to the first sequence. The second sequence may be quantitatively amplified, preferably using the same as that used to amplify the first sequence. The second sequence (ending with the 3′ most nucleotide of the sequence) is preferably located about 100 or more nucleotides to the 5′ end of the first sequence. Locations of about 150, about 175, about 200, about 225, about 250, about 275, about 300, about 325, about 350, about 375, about 400, or more to the 5′ end of the first sequence may also be used.

In alternative embodiments, the second sequence may be closer to the first sequence, such as separation by less than about 100 nucleotides, less than about 80 nucleotides, less than about 60 nucleotides, less than about 50 nucleotides, less than about 40 nucleotides, or less than about 20 nucleotides. In yet further embodiments, the second sequence may contain all or part of the first sequence. Stated differently, the first and second sequences overlap. This includes, but is not limited to, embodiments where the whole of the first sequence is present in the second sequence. Alternatively, the second sequence may contain, starting from the 5′ end of the first sequence, about 90% or less, about 80% or less, about 70% or less, about 50% or less, about 40% or less, about 30% or less, about 20% or less, or about 10% or less of the first sequence.

The second amplified sequence may be of any length, including those less than about 600 nucleotides, less than about 550 nucleotides, less than about 500 nucleotides, less than about 450 nucleotides, less than about 400 nucleotides, less than about 350 nucleotides, less than about 300 nucleotides, less than about 250 nucleotides, less than about 200 nucleotides, less than about 150 nucleotides, less than about 100 nucleotides, less than about 90 nucleotides, less than about 80 nucleotides, less than about 70 nucleotides, less than about 60 nucleotides, and less than about 50 nucleotides in length. In some embodiments, the lengths of the first and second sequence as amplified are the same or within about 5%, about 10%, about 20%, or about 30% of each other.

Where quantitative PCR is used in the practice of the invention, the Ct values from the amplifications of the first and second sequences may be used as a quantitative indicator of the amount of the amplified sequence upon comparison to a standard curve obtained by amplification of a control RNA sample. The control sample serves as the template which enables quantitation of the material amplified from a biological sample. The quantitative amplification method used to amplify RNA from the control sample is preferably the same as that used to amplify the first sequence of said RNA molecule. In some embodiments, the length of the amplified portion of the control RNA sample is the same as the length of the first and/or second sequence. In other embodiments, the amplified portion of the control RNA sampled is within about 5%, about 10%, about 20%, or about 30% of the length of the first and/or second sequence.

The invention also provides for the comparison of the amount of the first amplified sequence to the amount of the second amplified sequence as an indicator of RNA quality in the sample from which the assessed RNA was obtained. The comparison may be made by any suitable means. In some embodiments, the comparison may be made via a ratio of the amount of the first amplified sequence to the amount of the second amplified sequence. A low amount of the second sequence relative to the first sequence indicates that the RNA in the sample is more degraded (less intact) in comparison to situations where a high amount of the first sequence relative to the second sequence indicates that the RNA is more degraded (less intact). Stated differently, the comparison may be of the calculated quantity of the amount of RNA (number of RNAs to serve as templates) in the sample for detection of the first sequence versus the calculated quantity of the amount of RNA in the sample for detection of the second sequence. The invention includes comparisons expressed as a ratio of the amount of first (or 3′) amplicon, optionally in nanograms, to the amount of the second (or 5′) amplicon, in nanograms. Alternatively, the comparison may be by determination of the difference between the two amounts. The comparison may also be of the difference in Ct values for the amplification of the two amplicons.

Without being bound by theory, and offered to improve the understanding of the invention, it is believed that invention may also be used to determine or estimate the lengths of the longest intact RNA molecules in a biological sample. This is based on the decreasing ability to detect the second sequence (amplicon) as the number of intact RNA molecules decreases (or as the level of RNA degradation increases). Thus the locations of detectable second (or 5′) sequences, in relation to the location of the first (or 3′) sequence, provides an estimate of the length of RNA molecules containing the two sequences.

In alternative embodiments of the invention, the ratios may comprise the Ct values for the first amplified sequence and the second amplified sequence. Such ratios that indicate less degraded RNA in a sample are about 30 or less, about 25 or less, about 20 or less, about 15 or less, about 14 or less, about 13 or less, about 12 or less, about 11 or less, about 10 or less, about 9 or less, about 8 or less, about 7 or less, about 6 or less, about 5 or less, about 4 or less, or about 3 or less. Ratios that indicate more highly degraded RNA in a sample are about 35 or more, about 40 or more, about 50 or more, about 60 or more, about 70 or more, or about 80 or more.

The invention also provides kits containing reagents for the practice of the methods disclosed herein.

### Definitions

A “sequence” or “gene sequence” as used herein is a nucleic acid molecule or polynucleotide composed of a discrete order of nucleotide bases. The term includes the ordering of bases that encodes a discrete product (i.e. “coding region”), whether RNA or proteinaceous in nature, as well as the ordered bases that precede or follow a “coding region”. Non-limiting examples of the latter include 5′ and 3′ untranslated regions of a gene.

The terms “correspond” or “correspondence” or equivalents thereof refer to the relationship between nucleotides in separate nucleic acid molecules. The terms include at least the relationship between the nucleotides found in an RNA molecule, such as a polyadenylated RNA molecule, and those of the corresponding cDNA molecule reverse transcribed from said RNA, or polyadenylated RNA, molecule. The correspondence may be on a nucleotide to nucleotide basis between molecules, such as those of the same “strand” in a double stranded molecule, as well as on a nucleotide to nucleotide basis between two complementary strands of one double stranded molecule.

A “polynucleotide” is a polymeric form of nucleotides of any length, either ribonucleotides or deoxyribonucleotides. This term refers only to the primary structure of the molecule. Thus, this term includes double- and single-stranded DNA and RNA.

The term “amplify” is used in the broad sense to mean creating an amplification product that can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence.

Methods for amplifying MRNA are generally known in the art, and include reverse transcription PCR (RT-PCR) and those described in U.S. Pat. No. 6,794,141, which is hereby incorporated by reference in its entirety as if fully set forth. Another method which may be used is quantitative PCR (or Q-PCR). Such methods would utilize primers that are complementary to portions of a sequence to be amplified, where the primers are used to prime nucleic acid synthesis.

The term “label” or derivatives thereof refers to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means. In QPCR, labels include those which bind double stranded nucleic acids to result in a detectable signal as well as sequence specific (e.g. molecular beacon) probes that are converted into a detectable form during the QPCR reaction.

As used herein, a “biological sample” refers to a sample of tissue or fluid isolated from an individual or other biological source, including but not limited to, for example, blood, plasma, serum, spinal fluid, lymph fluid, the external sections of the skin, respiratory, intestinal, and genitourinary tracts, tears, saliva, milk, cells (including but not limited to blood cells), tumors, and organs. The individual is preferably human, and may be afflicted with, suspected of being afflicted with, or at risk of developing, a disease or unwanted condition. Such samples are primary isolates (in contrast to cultured cells) and may be collected by any non-invasive means, including, but not limited to, ductal lavage, fine needle aspiration, needle biopsy, the devices and methods described in U.S. Pat. No. 6,328,709, or any other suitable means recognized in the art. Alternatively, the “sample” may be collected by an invasive method, including, but not limited to, surgical biopsy. A sample of the invention may also be one that has been formalin fixed and paraffin embedded (FFPE) or freshly frozen.

The term “biological source(s)” as used herein refers to the sources from which polynucleotides are derived. The source can be any form of “biological sample” as described above, including but not limited to, cell, tissue or fluid. “Different biological sources” can refer to different cells, tissues or organs of the same individual, or cells, tissues or organs from different individuals of the same species, or cells, tissues or organs from different species, including cells or tissues that have been maintained in vitro or ex vivo. The term may also refer to cells, especially human cells, such as those that are malignant or otherwise associated with cancer, especially breast cancer; and cells that are laser-captured (laser capture microdissection) from fixed tissues from model organisms of human diseases or actual human tissue (postmortem or biopsy material).

A “portion” or “region,” used interchangeably herein, of a polynucleotide or oligonucleotide is a contiguous sequence of 2 or more bases. In other embodiments, a region or portion is at least about any of 3, 5, 10, 15, 20, 25 contiguous nucleotides.

The term “3′” (three prime) generally refers to a region or position in a polynucleotide or oligonucleotide that is 3′ (downstream) from another region or position in the same polynucleotide or oligonucleotide.

The term “5′” (five prime) generally refers to a region or position in a polynucleotide or oligonucleotide that is 5′ (upstream) from another region or position in the same polynucleotide or oligonucleotide.

The term “3′-DNA portion,” “3′-DNA region,” “3′-RNA portion,” and “3′-RNA region,” refer to the portion or region of a polynucleotide or oligonucleotide located towards the 3′ end of the polynucleotide or oligonucleotide, and may optionally include the 3′ most nucleotide(s) or moieties attached to the 3′ most nucleotide of the same polynucleotide or oligonucleotide.

The term “5′-DNA portion,” “5′-DNA region,” “5′-RNA portion,” and “5′-RNA region,” refer to the portion or region of a polynucleotide or oligonucleotide located towards the 5′ end of the polynucleotide or oligonucleotide, and may optionally include the 5′ most nucleotide(s) or moieties attached to the 5′ most nucleotide of the same polynucleotide or oligonucleotide.

“Expression” and “gene expression” include transcription and/or translation of nucleic acid material. An expressed sequence or gene is one that is expressed in a cell of a biological sample of the invention. Expressed sequences also refers to the RNA molecules, including polyadenylated RNA molecules, that are produced (or “expressed”) from a template nucleic acid.

As used herein, the term “comprising” and its cognates are used in their inclusive sense; that is, equivalent to the term “including” and its corresponding cognates.

Unless defined otherwise all technical and scientific terms used herein have the same meaning as commonly understood to one of ordinary skill in the art to which this invention belongs.

It must be noted that as used in this specification and the appended claims, the singular forms “a”, “an” and “the” include corresponding plural references unless the context clearly dictates otherwise.

### BRIEF DESCRIPTION OF THE DRAWINGS

FIG. 1 illustrates two possible embodiments (or designs) for the practice of the invention. Various lengths of a RNA sequence (or the corresponding cDNA) are represented as horizontal lines denoted as a, b, c, and d. Above those lines are an illustration of one embodiment of the invention wherein the first sequence (or amplicon) is defined by the forward and reverse primers for PCR denoted as “F” and “R” respectively. The same reverse primer “R” is also used in combination with other possible forward primers, denoted “F1”, “F2”, and “F3” which define three possible second sequences (or amplicons) which contain the first sequence in its entirety given the use of a common reverse primer. A single sequence specific probe, denoted by “probe” and indicated by the short thick line, may be used to detect all the possible amplicons.

Below lines a through d is an illustration of an embodiment wherein no overlap between the first and second sequences occurs. The first sequence (or amplicon) is denoted by a forward and reverse primer as explained above. It is detectable by a sequence specific probe denoted “probe” and shown as a short thick line. Two possible second sequences (amplicons) are indicated as “Taqman 1” and “Taqman 2”. Each of these two possible second sequences (amplicons) is detectable by a sequence specific probe shown as a short thick line below each of “Taqman 1” and “Taqman 2”.

FIG. 2 shows a schematic representation of the β-actin mRNA, full length of 1761. The positions of a 5′ amplicon and a 3′ amplicon are shown.

FIG. 3, top, shows the mean fluorescence intensity of signals of amplified and labeled molecules obtained from RNA in the samples and hybridized to a microarray. The lower portion shows the 3′/5′ ratio for the same samples.

FIG. 4 shows three profiles of samples with relatively intact RNA.

FIG. 5 shows three additional profiles of RNA samples.

FIG. 6 shows three profiles of samples with relatively degraded RNA.

FIG. 7 shows an exemplary PCR instrumentation protocol.

FIG. 8 illustrates exemplary thermal cycler conditions for β-actin amplicons on the 7900HT instrument.

FIG. 9 shows standard curves from the 1355 and 1650 amplicons on the 7900HT instrument.

FIG. 10 shows results from gel electrophoresis of fragmented RNA.

### DETAILED DESCRIPTION OF MODES OF PRACTICING THE INVENTION

The invention provides methods of assessing RNA quality by comparison of the amount of material amplified from one portion of an expressed sequence to the amount of material amplified from a second portion of the same expressed sequence. The invention may thus be practiced with the use of two amplicons, defined as the sequences of the two regions that are amplified. “Amplicon” also refers to the product of a PCR or LCR reaction, or the nucleic acid molecule synthesized in the reaction.

The invention thus provides for the determination of the overall quality of an RNA preparation from a biological sample by analysis of two sequences from a representative RNA molecule in the preparation. Of course the analysis may also be used as a direct indicator of the level of RNA degradation of the representative RNA molecule per se. Moreover, the invention may optionally be practiced with the analysis of first and second sequences, as described herein, from more than one representative RNA molecule in the preparation. This alternative allows for a greater level of confidence in the assessment of RNA degradation by providing information on more than one RNA species.

In one embodiment, the invention is practiced by first preparing a biological sample obtained from a subject. The sample is optionally sectioned, stained, and/or microdissected to obtain particular cells for analysis. RNA is then extracted/isolated followed by its analysis, optionally beginning with cDNA synthesis. The prepared cDNA may be of a plurality of RNA species or of particular subsets of RNA types. As a non-limiting example, reverse transcription using oligo-dT or poly-dT containing primers will produce cDNAs of polyadenylated RNA molecules. The cDNAs will contain sequences corresponding to the polyA tail of the RNA molecules. Alternatively, use of random primers, such as those of 6, 7, 8, 9, 10, 11, 12 or more random nucleotides, can be used to produce a population of random cDNA molecules. As a further possibility, one or more specific primers may be used to produce only a limited population of cDNAs corresponding to RNA molecules with sequence complementarity to the specific primer(s) used.

The cDNA may then be used for quantitative, or real time, PCR of two regions of a particular sequence known or suspected to be expressed as an RNA molecule in the sample. The particular sequence may be any expressed sequence, and in some embodiments, it is a sequence of a reference gene known to be expressed as an RNA in the biological sample. The amount of amplification of the two regions, where the first may be viewed as a 3′-region and the second as a 5′ region relative to the cDNA, and thus corresponding polyadenylated RNA, is then used to assess the extent of degradation of the RNA. The assessment may be made by determination of a ratio, or metric, of the amounts of the two amplified regions based upon a comparison to amplification of a control RNA, such as, but not limited to, Universal RNA (uRNA from Stratagene).

The invention thus provides a method of assessing the quality of RNA in a biological sample based on the description herein. The method comprises preparing a population of cDNA molecules from (expressed) RNA in said biological sample, and quantitatively amplifying a first sequence and a second sequence of a cDNA molecule in said population to produce first and second amplicons, respectively. The second sequence is 5′ from the first sequence. The amounts of the two amplicons are then compared to determine the level of RNA degradation, or the level of RNA intactness, where a low amount of the first amplicon relative to the second amplicon indicates that the RNA in said sample is less degraded (or more intact) than where a high amount of the first amplicon relative to the second amplicon is found (and thus indicating that the RNA in the sample is more degraded and less intact).

The RNA of the biological sample may be expressed from any nucleic acid present in the sample, and includes polyadenylated RNA molecules. A polyadenylated RNA in the sample may be used to prepare cDNA(s) for use in the invention. However, the preparation of cDNA may be omitted where quantitative detection of the first and second sequences can be made via the RNA without conversion to cDNA.

The first and second sequences of the invention may be separate such that they do not overlap. Alternatively, the method of claim 1 wherein said second amplicon contains sequences present in said first amplicon because the sequences of the two amplicons overlap. In some embodiments, the entirety of the sequence of the first amplicon is present in the second amplicon.

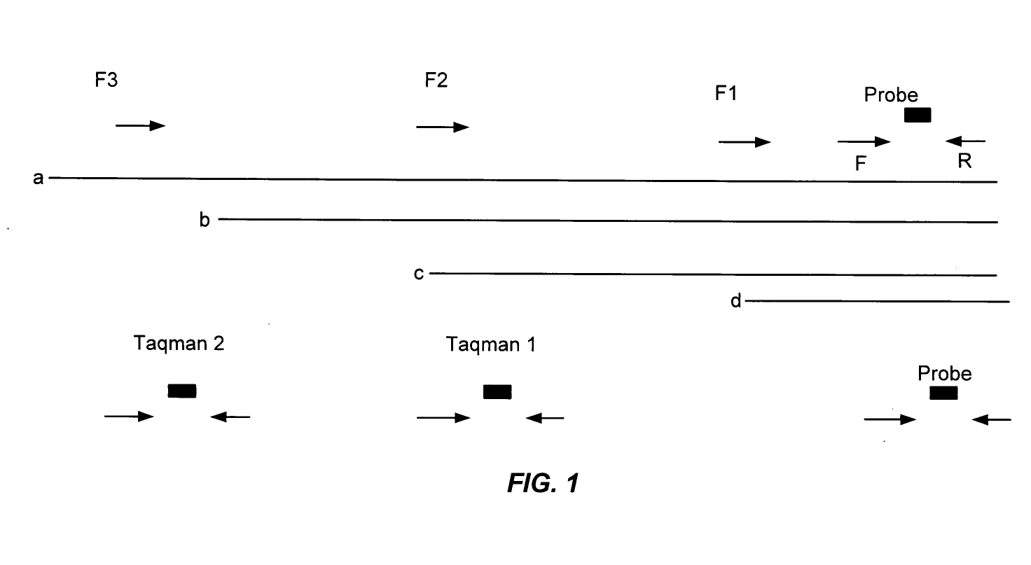

Non-limiting alternative embodiments of the invention are shown in FIG. 1, which illustrates two non-limiting embodiments of the invention. The first, shown in the upper portion of the Figure, utilizes first and second sequences that overlap. Because a single sequence specific probe is illustrated for use in detecting both the first and second sequences, this embodiment of the invention may be practiced via two separate reactions: one including the use of forward primer F and another including use of forward primers F1, F2, F3 or combinations thereof. This approach would also be used where a sequence independent probe which binds double stranded nucleic acids (such as SYBR Green or the like) is used in place of a sequence specific probe.

This embodiment of the invention may also be practiced in a multiplex mode wherein sequence specific probes for each of the amplicons defined by one of F, F1, F2, and F3 and R are used. As would be appreciated by the skilled person, where the RNA is degraded such that the population of intact longer RNAs, such as “a” in FIG. 1 are rare, a larger amount of the first amplicon, defined by primers F and R, will be produced relative to a second amplicon defined by primers F3 and R. Additionally, the skilled artisan will appreciate that the use of the amplicon defined by primers F and R as the first amplicon is arbitrary because it is simply 3′ to the other three amplicons shown (and defined by F1 and R, F2 and R, and F3 and R). Thus as a non-limiting example, the amplicon defined by F1 and R may be used as the first amplicon relative to the amplicon defined by F2 and R, or F3 and R, each of which may be used as the second amplicon relative to F1 and R.

Another embodiment is shown on the lower portion of FIG. 1, wherein the first amplicon (defined by primers F and R) and second amplicon (indicated by Taqman 1 or Taqman 2) do not overlap. As noted above, where the RNA is degraded such that the population of RNAs of length “a” in FIG. 1 are rare, a larger amount of the first amplicon, defined by primers F and R, will be produced relative to a second amplicon identified as Taqman 1 or Taqman 2. Moreover, and analogous to the above, the skilled artisan will appreciate that the use of the amplicon defined by primers F and R as the first amplicon is arbitrary because it is simply 3′ to the other two amplicons shown (identified as Taqman 1 and Taqman 2). Therefore, the Taqman 1 amplicon may be used as the first amplicon relative to the Taqman 2 amplicon as shown in the Figure.

The selection of primers to use in the practice of the invention may be by any suitable means known in the art. In some embodiments, the primers are selected such that their sequence “straddles” an exon-exon junction in a gene sequence. This permits the primer to be specific for spliced sequences, such as those in processed RNA (and thus the corresponding cDNA). Such primers are less likely to result in the amplification of contaminating genomic DNA material present in the biological sample.

In embodiments using QPCR, the amplification of the first and second sequences can be monitored to produce a first Ct value for the amplification of the first amplicon and a second Ct value for the amplification of the second amplicon. These first and second Ct values may be compared to assess the level of RNA degradation as described herein. As a non-limiting example, the difference between the two Ct values (or ACt) may be as the metric.

In other embodiments, the Ct values may be converted to the amount of amplified material (expressed in terms relating to mass) before comparison. In some embodiments, the amount of material is expressed in terms of grams or nanograms, and a ratio of the amount of RNA corresponding to the first amplicon and the amount of RNA corresponding to the second amplicon is used as the metric.

In embodiments where determination of a ratio, or metric, of the amounts of the two amplified regions based upon a comparison to amplification of a control RNA is used, the comparison may be performed by PCR using a dilution series of the control RNA. As a non-limiting example, dilutions equivalent to 100 ng, 10 ng, 1 ng, and 0.1 ng of the control RNA can be used in QPCR to provide a standard curve of Ct values versus RNA quantity. This curve can then be used to determine the amount of each amplicon that has been amplified by intrapolation.

In some embodiments of the invention, the biological sample is an FFPE sample wherein the RNA is degraded such that the reverse transcription process to obtain cDNA results in cDNAs that are truncated in length due to RNA degradation as well as base modification resulting from the fixation process.

Uses of the present invention include providing the ability to assess RNA quality more accurately than previously possible. This provides an added advantage where RNA samples which would have been considered too degraded, based on previous assessment techniques, for gene expression analysis can be identified as sufficiently intact for use, such as in the methods described in U.S. patent application Ser. No. 10/329,282, filed Dec. 23, 2002 and PCT application PCT/US03/32345, filed Oct. 10, 2003, which are hereby incorporated by reference as if fully set forth.

The materials for use in the methods of the present invention are ideally suited for preparation of kits produced in accordance with well known procedures. The invention thus provides kits comprising agent(s) for the assessment of RNA quality. Such kits optionally comprising the agent(s) with an identifying description or label or instructions relating to their use in the methods of the present invention, is provided. Such a kit may comprise containers, each with one or more of the various reagents (typically in concentrated form) utilized in the methods, including, for example, primers, probes, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes of the present invention (e.g., appropriate length poly(T)). A set of instructions will also typically be included.

The methods provided by the present invention may also be automated in whole or in part.

Having now generally described the invention, the same will be more readily understood through reference to the following examples which are provided by way of illustration, and are not intended to be limiting of the present invention, unless specified. Thus while the following discussion includes the use of a human β-actin MRNA as an exemplar, it will be appreciated by the skilled artisan that the invention may be practice with virtually any known sequence that is expressed, regardless of whether it is expressed as a polyadenylated RNA molecule.

### EXAMPLES

### Example 1

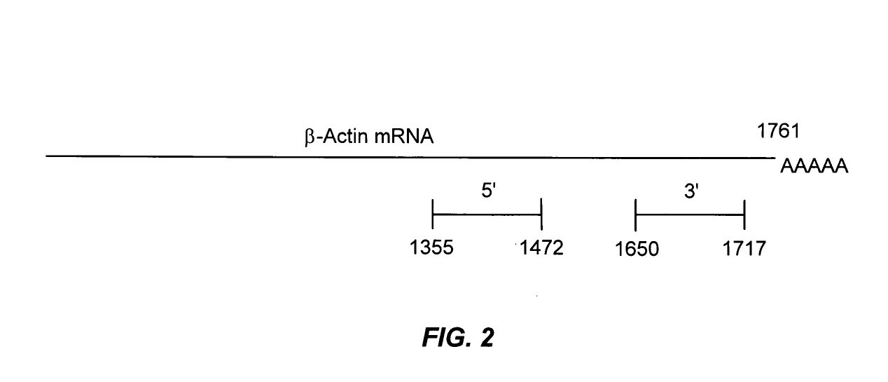

Quality assessment process Quantitative PCR was conducted based on amplicons located 100 and 400 nucleotides away from the 3′ end of the β-actin MRNA. See FIG. 2. The quantity of RNA is calculated based on Ct values from each amplicon. The ratio of the quantities of the 3′ (first) amplicon and the 5′ (second) amplicon are used as a metric for RNA quality assessment.

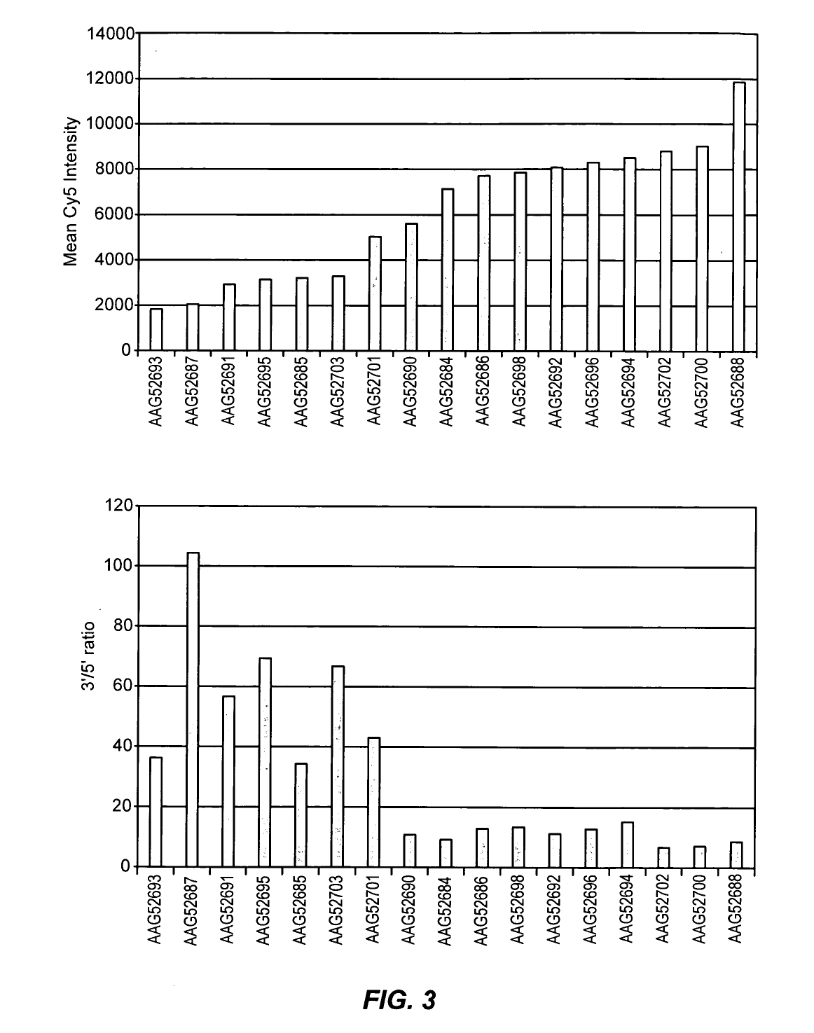

The 3′ to 5′ ratios of the respective amplicons from the β-actin MRNA for a variety of samples are shown in FIG. 3 in combination with the mean fluorescence intensity of signals of amplified and labeled molecules obtained from RNA in the samples and hybridized to a microarray. As FIG. 3, the higher the 3′/5′ ratio the more degraded the sample. This is evident in the CY5 signal (top graph) which shows that with larger 3′/5′ ratios (bottom graph) comes lower mean intensities of the CY5 signal on the array.

### Example 2

### Comparisons to Bioanalyzer Profiles

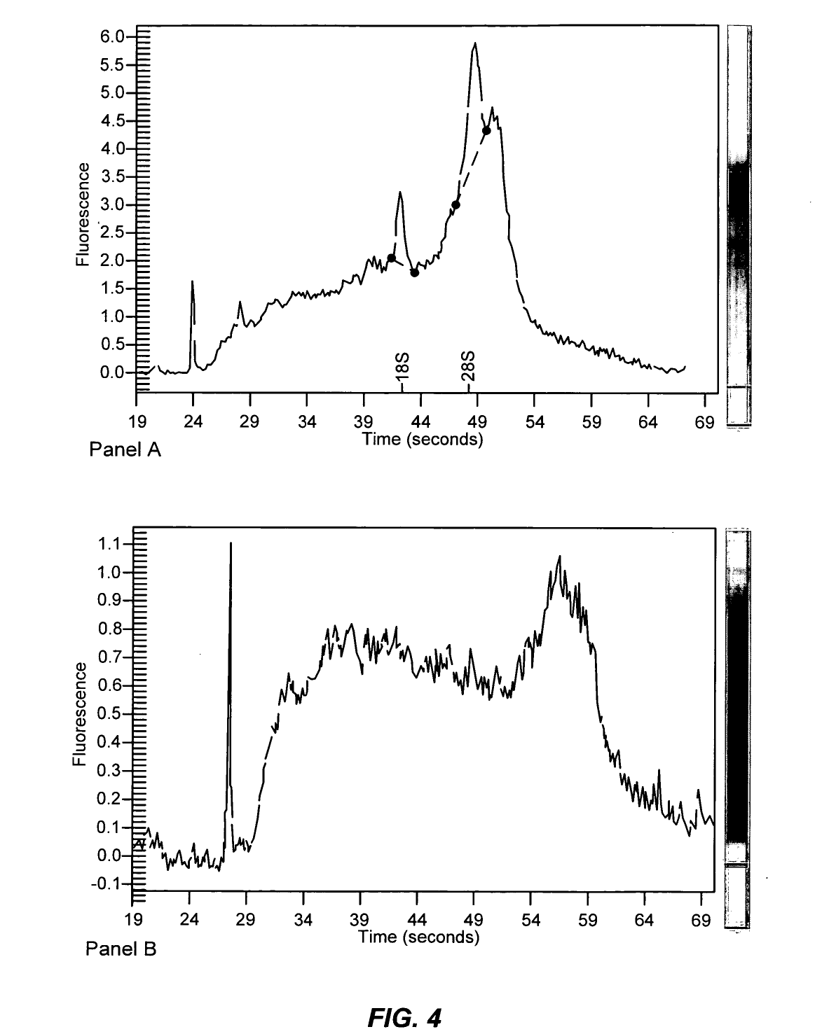

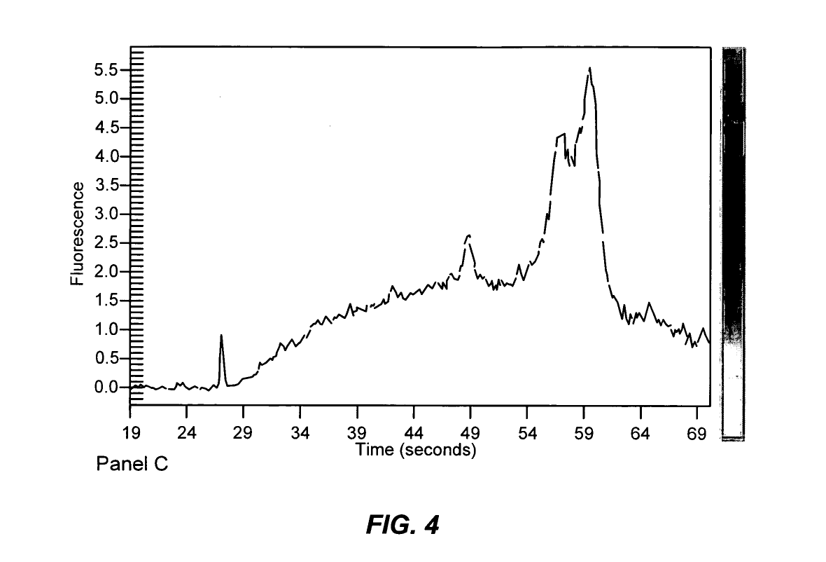

Bioanalyzer profiles of total RNA isolated from formalin-fixed and paraffin-embedded (FFPE) RNA can look very different from conventional profiles seen from good quality total RNA from frozen tissue. While it is sometimes possible to see 18S/28S ribosomal RNA bands in FFPE RNA, it is more likely that the majority of samples will not show clear 18S/28S peaks. It is possible however, to judge the quality of RNA from Bioanalyzer profiles in many cases (see FIG. 4 and their associated QRT-PCR results). The profiles also provide a rough estimate of the amount of RNA isolated per unit area. In certain cases, the quality of RNA may need to be confirmed using additional assays such as QRT-PCR.

The profiles in FIG. 4 are generated from 1 ng of FFPE RNA following RNA extraction isolation (using the Paradise™ Reagent System from Arcturus Bioscience, Inc.) from macro-dissected tumor area within the tissue sections. These runs were performed on an Agilent Bioanalyzer Picochip.

FIG. 4, Panels A-C show examples of good quality FFPE RNA. In Panel A, the RNA from an FFPE sample was assessed as good quality, 18S/28S visible, good RNA yield. The β-actin ratio (1650 amplicon/1355 amplicon) was 3.7 (see Example 3 below). In Panel B, the RNA from an FFPE sample was assessed as good quality, 28S visible, low RNA yield. The β-actin ratio (1650 amplicon/1355 amplicon) was 8.6. In Panel C, the RNA from an FFPE sample was assessed as good quality, 18S/28S visible, good RNA yield. The β-actin ratio (1650 amplicon/1355 amplicon) was 10.8.

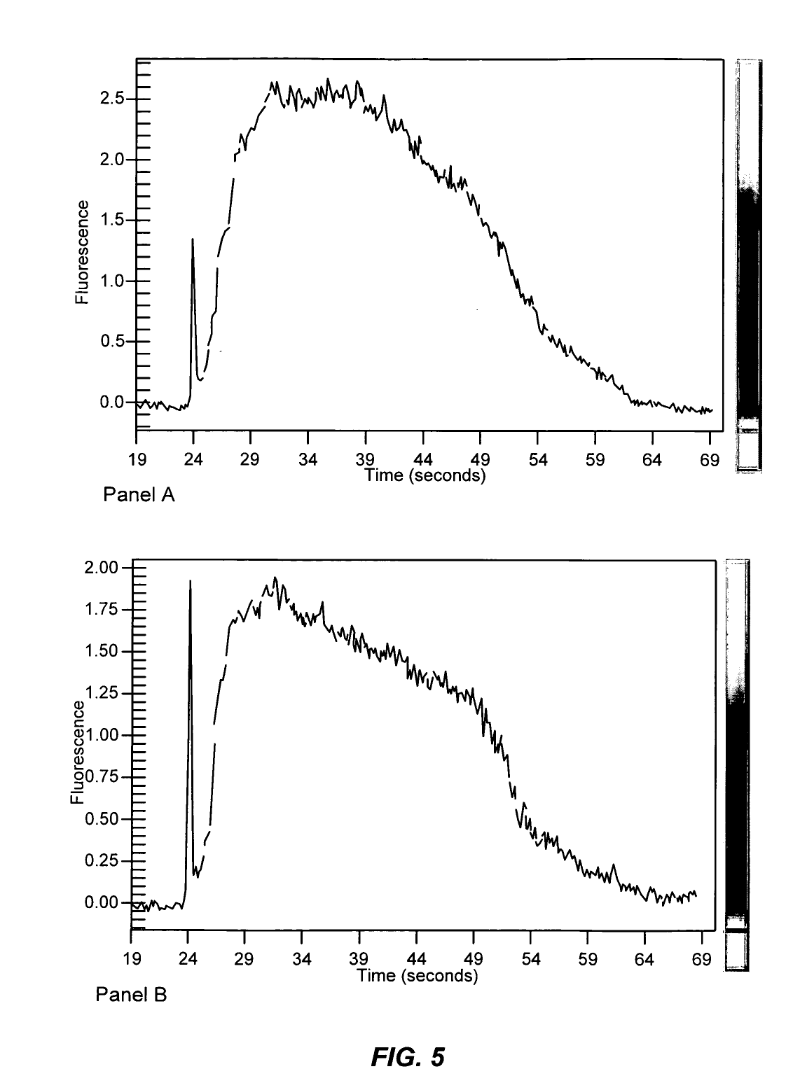

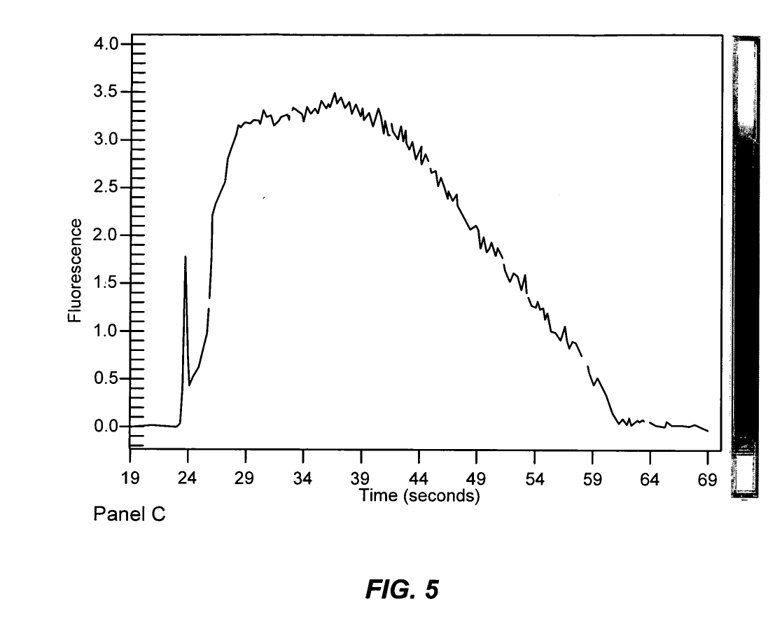

FIG. 5, Panels A-C are examples of Bioanalyzer profiles in which use of the present invention would be advantageous because profiles similar in appearance have very different QRT-PCR results as determined by use of the present invention. In Panel A, the RNA from an FFPE sample was assessed as suspect, 18S/28S not visible, moderate RNA yield. The β-actin ratio (1650 amplicon/1355 amplicon) was 9.5. In Panel B, the RNA from an FFPE sample was assessed as suspect, 18S/28S not visible, moderate RNA yield. The β-actin ratio (1650 amplicon/1355 amplicon) was 13.0. In Panel C, the RNA from an FFPE sample was assessed as suspect, 18S/28S not visible, moderate RNA yield. The β-actin ratio (1650 amplicon/1355 amplicon) was 34.3. As evident from the above, use of the invention was able to identify the RNA of FIG. 5, Panel C as more degraded than would be expected based on the Bioanalyzer profile alone.

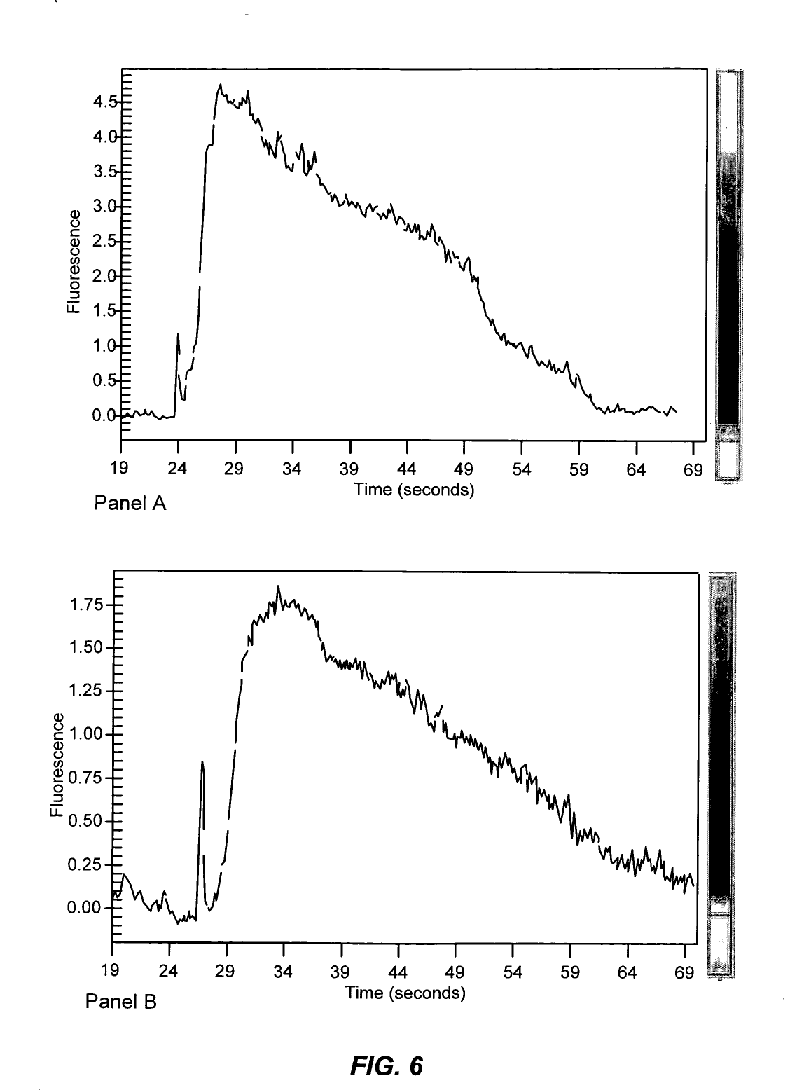

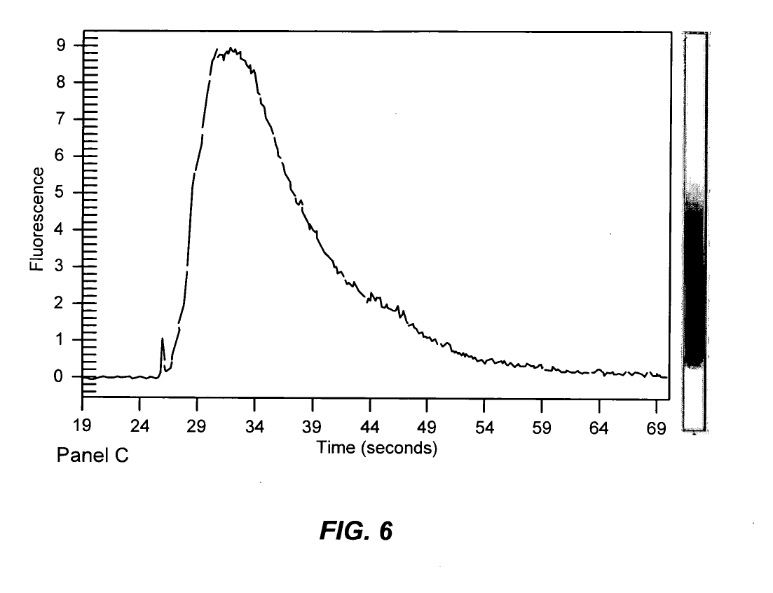

FIG. 6, Panels A-C are examples of Bioanalyzer profiles of poor quality FFPE RNA which is confirmed by QRT-PCR results via use of the present invention. All three profiles show a predominance of low molecular weight RNA, suggesting poor quality of RNA. In Panel A, the RNA from an FFPE sample was assessed as poor with good RNA yield. The β-actin ratio (1650 amplicon/1355 amplicon) was 66.5. In Panel B, the RNA from an FFPE sample was assessed as poor with moderate RNA yield. The β-actin ratio (1650 amplicon/1355 amplicon) was 56.9. In Panel C, the β-actin ratio (1650 amplicon/1355 amplicon) was 68.9.

### Example 3

### Exemplary Protocols Using Two cDNA Methods and Two PCR Instruments

This protocol provides a method of assessing the quality of the RNA in formalin fixed paraffin embedded (FFPE) samples. Total RNA from a 0.5 cm×0.5 cm tissue scrape was processed following the protocol provided with the Paradise™ Reagent System or the Invitrogen Superscript First-Strand Synthesis Kit. A portion of this RNA is used to perform a test according to the present invention. The test involves a reverse transcription of the total RNA followed by quantitative PCR. In addition to the sample a control RNA is also be conducted. This control sample serves as the template for a standard curve which permits quantitation of the sample material.

Two primer sets are used in this protocol. The ratio of the RNA quantity determined using two different primer sets is an indicator of the quality of RNA in the sample. The following includes protocols for use with the Light Cycler Real Time Instrument as well as the ABI 7900 PCR System. Modifications to the protocol for use with other real time platforms and instrumentation can be made.

Reagents used: Primer 1 (Included with Paradise Reagent System); RNase free water (Invitrogen 10977-015); Universal Human Reference RNA (Stratagene, 740000); Invitrogen SuperScript First-Strand Synthesis system for RT-PCR (11904-018); PolyI (Sigma, P4154); LightCycler DNA SYBR Green kit (Roche, 2158817); BD Taqstart Antibody (BD-Clontech, 639251); and Uracil-DNA Glycosylase (Roche, 1444646).

Primer sequences used:

For the 3′ amplicon (hence 1650):

| | |
|---|---|
| HBAC1650 | TCCCCCAACTTGAGATGTATGAAG |
| HBAC1717 | AACTGGTCTCAAGTCAGTGTACAGG |

For the 5′ amplicon (hence 1355):

| | |
|---|---|
| HBAC1355 | ATCCCCCAAAGTTCACAATG |
| HBAC1472 | GTGGCTTTTAGGATGGCAAG |

Equipment used: Thermal Cycler and Lightcycler (Roche, 2011468) or ABI 7900 PCR System.

RNA extracted from FFPE tissue scrape samples according to the Paradise process were in a final volume of 70 μl. 8 μl was used for the following analysis. Alternatively, about 25 ng RNA estimated from the OD 260 estimation may be used.

In the protocol example below this sample is called the testing sample (T). In parallel to the testing sample, a control universal RNA (100 ng/μl) is processed as well. This control RNA serves as the quantitation standard used in making the standard curve and will be referred to as the control RNA (C1), this control will be used in every experiment.

There are two experimental steps (however, the invention may also be practiced with a “one tube” process where the product from reverse transcription is directly used for QPCR):
Step 1. Reverse Transcription reaction with a Oligo dT based primer (P1) in a thermal cycler.
Step 2. Quantitative PCR using two sets of human actin primers (1650 and 1717, 1355 and 1472) to produce two amplicons in PCR instrument.

The results from the tested sample(s) are compared to a standard curve generated in the same experiment using a serial diluted cDNA from human universal RNA to determine the sample RNA yield. The ratio of the RNA yield obtained from the two sets of the PCR primers is an indicator of the RNA quality.

The experimental design illustrated below is for three testing samples, one blank and one uRNA control. This serves as an example, and samples included can be modified accordingly. This summary provides an overview of the procedure and is followed by a detailed protocol.

In five tubes, set up RT reactions for three testing samples, one blank control and one uRNA standard:

TABLE 1

| | Tube | |
|---|---|---|
| Tube # | name | Sample |
| 1 | T1 | Testing 1, 25 ng/8 ul FFPE sample |
| 2 | T2 | Testing 2, 12.5 ng/8 ul FFPE sample |
| 3 | T3 | Testing 3, 6.25 ng/8 ul FFPE sample |
| 4 | B | Blank |
| 5 | C1 | 100 ng/8 ul uRNA |

After the RT reaction, generate serial dilution of cDNA from uRNA (C1) (see Table 3 C2, C3, C4 in tube 6, 7, 8). Set up 8 PCR reactions with samples 1-8 for the PCR instrument using primers 1650 and 1717. Obtain Ct for all reactions.

Set up another set of 8 PCR reactions with sample 1-8 for the PCR instrument using primers 1355 and 1472. Obtain Ct for all reactions.

Quality Control (QC) Protocol:

1. Reverse transcription reaction (RT)
a. Thaw Primer 1 (provided with Paradise System) thoroughly and mix.
b. Set up one RT reaction for each testing sample (T), one for a blank, and one for the uRNA standard (C1).
c. Add 1 μl Primer1 to each tube.
d. Add 8.0 μl of one testing sample in corresponding tube. Add 8.0 μl of water in the blank tube. Add 8 μl of the uRNA standard in tube 5. Mix thoroughly by flicking the tube and spin down.
e. Incubate at 70° C. for 1 hour then chill the samples to 4° C. for at least one minute. Keep the tubes at 4° C. before proceeding to step f.

f. RT synthesis mix. Thaw 0.1 M DTT, 10× RT reaction buffer, 10 mM dNTP, 25 mM MgCl₂, and RNaseOUT from the Invitrogen kit (alternatively, use the cDNA synthesis protocols from the Paradise System). Each component needs to be thawed thoroughly with all solids dissolved and then maintained at 4° C. Make the RT reaction mix in a 0.5 ml tube in the order indicated below (Table 2). The following table lists the amount of reagents for each reaction in the middle column. Make the master mix for reactions by multiply the number of the reactions plus one (n+1) to the volume of each component. For the example experiment design, n+1=6. The volume of each reagent for the example experiment is shown on the last column of Table 2. Mix and spin briefly.

TABLE 2

| | | Volume for example |
|---|---|---|
| Components | Volume | n + 1 = 6 |
| 10 mM dNTP | 1 ul | 6 |
| 10x RT buffer | 2 ul | 12 |
| 25 mM MgCl2 | 4 ul | 24 |
| 0.1 M DTT | 2 ul | 12 |
| RNaseOUT | 1 ul | 6 |

g. Add 10 μl of the master mix to each reaction tube. Mix thoroughly by flicking the tube. Spin down.
h. Incubate at 42° C. for 2 min.
i. Add 1 μl of Superscript II RT to each tube. Incubate at 42° C. for 50 min.
j. Terminate the reaction at 70° C. for 15 min.
k. Chill the sample to 4° C. for at least one minute.
l. The reaction mix can be stored at −20° C. before proceeding to the PCR reaction.

2. Quantitative PCR on Lightcycler

a. Make a serial dilution of cDNA from the uRNA standard (tube 5, after the RT reaction) as shown in the following table:

TABLE 3

| | Tube | 10 ng/ul | |
|---|---|---|---|
| Tube # | name | poly I (ul) | cDNA solution |
| 6 | C2 | 18 | 2 ul #5 |
| 7 | C3 | 18 | 2 ul #6 |
| 8 | C4 | 18 | 2 ul #7 |

b. Prepare a PCR mix using PCR instrument, DNA SYBR Green kit, Taqstart Antibody, and Uracil-DNA Glycosylase. For each PCR reaction (one cDNA sample with one pair of primers) make 18 μl of the mix as listed in Table 4. Multiply the amount of each component by the number of reactions plus one (n+1). Mix thoroughly by inverting the tube, and then spin briefly.

TABLE 4

| | | Volume for example, |
|---|---|---|
| Components | Volume (ul) | n + 1 = 9 |
| SYBR Green Master | 2 | 18 |
| BD Taqstart Antibody | 0.16 | 1.71 |
| 25 mM MgCl2 | 2.4 | 21.6 |
| Forward primer (50 uM) | 0.25 | 2.25 |
| Reverse Primer (50 uM) | 0.25 | 2.25 |
| Uracil-DNA Glycosylase | 1 | 9 |
| water | 11.94 | 107.46 |

c. For each reaction, add 18 μl of the PCR mix into a LightCycler capillary. Add 2 μl of the cDNA. In the example experiment, set up 8 capillaries for reaction 1 to 8. Spin the capillaries at 500 g for 5 second in their adaptor. Load the capillaries into LightCycler. Table 5 is a summary of the samples.

TABLE 5

| Tube # | Tube name | content |
|---|---|---|
| 1 | T1 | Testing sample 1 |
| 2 | T2 | Testing sample 2 |
| 3 | T3 | Testing sample 3 |
| 4 | B | Blank |
| 5 | C1 | 100 ng uRNA |
| 6 | C2 | 10 ng uRNA |
| 7 | C3 | 1 ng uRNA |
| 8 | C4 | 0.1 ng uRNA |

d. Run the LightCycler with corresponding programs. The programs for mer pairs 1650 and 1717 and 1355 and 1472 are listed below (Table 6a).

TABLE 6a

| | | Target | | Temp | |
|---|---|---|---|---|---|
| Primer set | | temp | Incubation | transition | Acqui- |
| +1650:1717 | | (° C.) | time | rate | sition |
| Step 1 | Denaturation | 95 | 1 min | 20 | none |
| Step 2 | Amplification | 95 | 0 sec | 20 | none |
| | −35 cycles | | | | |
| | | 60 | 5 sec | 20 | none |
| | | 72 | 10 sec | 20 | single |
| Step 3 | Melting | 95 | 0 sec | 20 | none |
| | | 65 | 10 sec | 20 | none |
| | | 99 | 0 sec | 20 | cont |
| Step 4 | Cooling | 40 | 1 min | 20 | none |

TABLE 6b

| | | Target | | Temp | |
|---|---|---|---|---|---|
| Primer set | | temp | Incubation | transition | Acqui- |
| +1355:1472 | | (° C.) | time | rate | sition |
| Step 1 | Denaturation | 95 | 1 min | 20 | none |
| Step 2 | Amplification | 95 | 0 sec | 20 | none |
| | −35 cycles | | | | |
| | | 58 | 5 sec | 20 | none |
| | | 72 | 10 sec | 20 | single |
| Step 3 | Melting | 95 | 0 sec | 20 | none |
| | | 65 | 10 sec | 20 | none |
| | | 99 | 0 sec | 20 | cont |
| Step 4 | Cooling | 40 | 1 min | 20 | none |

e. Obtain a Ct value for the 1650 primer set for the testing sample and the uRNA dilutions. In the example experiment, obtain Ct 1650 for sample 1 to 8.
f. Carry out the PCR reaction for the 2nd pair of primers. Start at step 2b, but this time making the PCR reaction mix with the second pair of primers. Obtain the Ct for the testing sample and for the uRNA dilutions. In the example experiment, obtain Ct 1355 for sample 1 to 8.

3. Quantification of the Input RNA
a. Plot the standard curve of log uRNA amount vs. Ct. For each pair of primers, one standard curve is generated.
b. Obtain the uRNA equivalent of the testing sample from the corresponding standard curve (Standard curve 1650, Standard curve 1355).
c. Use the uRNA equivalent from 1650 primer set to estimate the RNA quantity, use the ratio of RNA 1650/RNA 1355 to estimate the RNA quality.

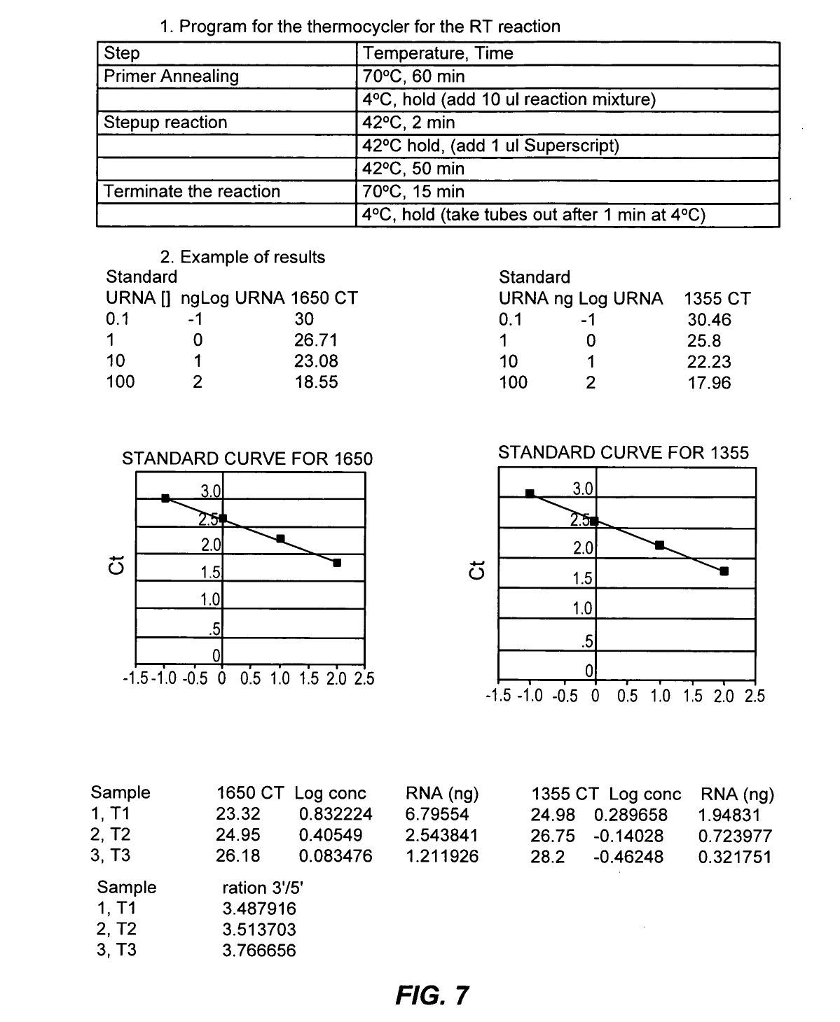

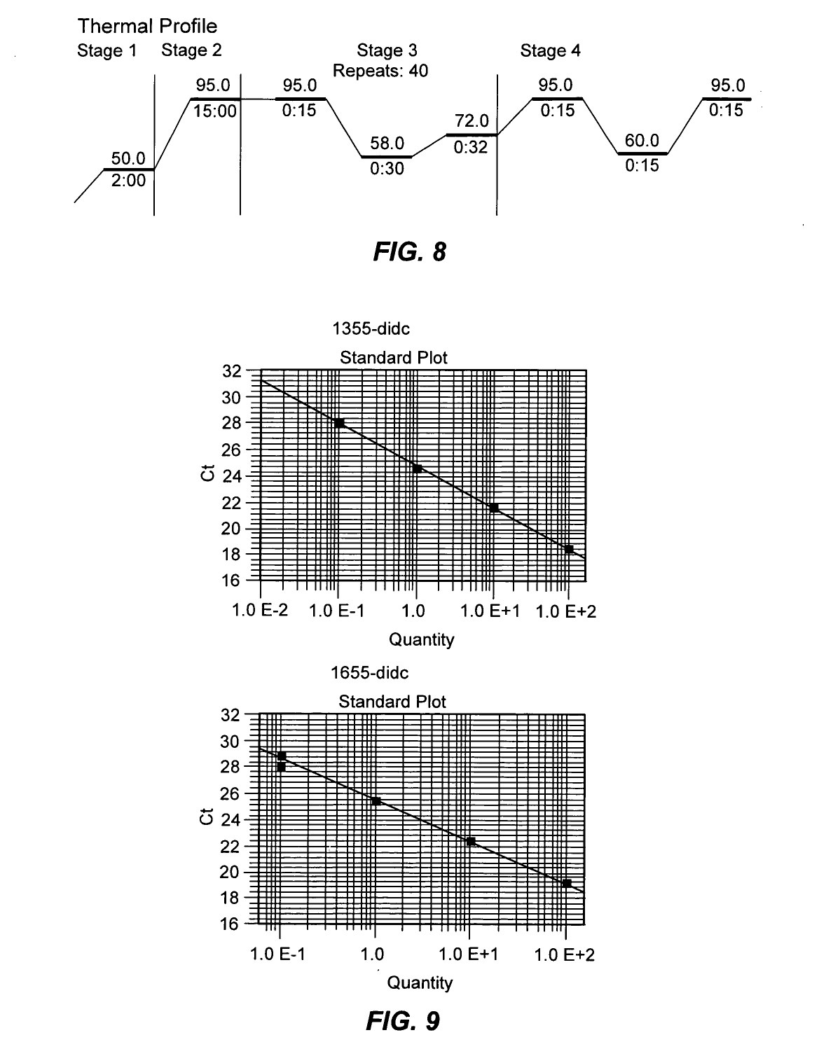

An exemplary instrumentation protocol is shown in FIG. 7. FIG. 8 illustrates exemplary thermal cycler conditions for β-actin amplicons on the 7900HT instrument. FIG. 9 shows standard curves from the 1355 and 1650 amplicons on the 7900HT instrument.

### Example 4

### Testing with Fragmented uRNA

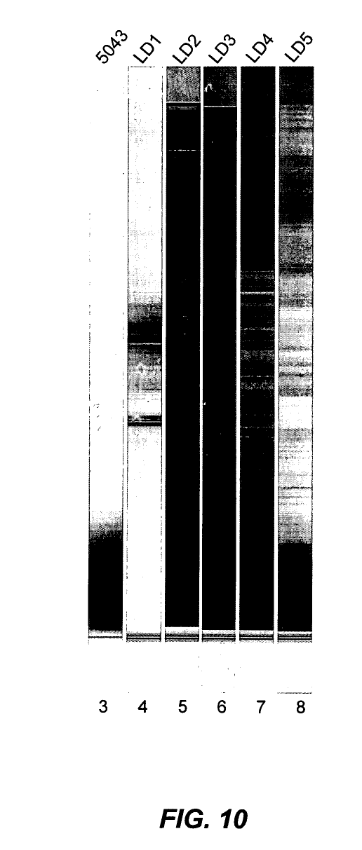

uRNA was fragmented via heat treatment for various times and an aliquot was analyzed by gel electrophoresis. See FIG. 10. Lanes 4-8 show the conditions of samples that have been heated for 0, 2, 4, 8, and 16 minutes, respectively.

Table 7 shows the results of detecting the 3′ and 5′ amplicons for β-actin (indicated as “RI”) MRNA in the same fragmented uRNA samples. The length of heat treatment is shown in the leftmost column, followed by five columns showing the Ct value for the 3′ amplicon, the Ct value for the 5′ amplicon, the calculated RNA amount (in nanograms) for the 3′ amplicon, the calculated RNA amount (in nanograms) for the 5′ amplicon, and the ratio of the 3′ to 5′ RNA amounts.

As shown, the amount of RNA for each amplicon decreases as the length of heating time increases. Moreover, after 2 and 4 minutes of heating, the presence of relatively undegraded RNA is still observed. After 8 minutes or more of heating, however, significant degradation is observed, along with significant losses in the amount of RNA, which can be used as an indicator in addition to the ratio of RNA amounts.

TABLE 7

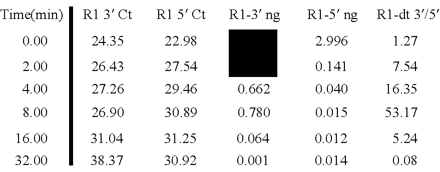

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of assessing the degree of RNA degradation in a biological sample, said method comprising
preparing a population of cDNA molecules from expressed RNA in said biological sample;
quantitatively amplifying a first sequence of a cDNA molecule in said population to produce a first amplicon;
quantitatively amplifying a second sequence from said cDNA molecule to produce a second amplicon, wherein said second sequence is 5′ to the first sequence;
comparing the amount of said first amplicon to the amount of said second amplicon, wherein a low amount of the second amplicon relative to the first amplicon indicates that the RNA in said sample is more degraded than wherein a high amount of the first amplicon relative to the second amplicon is found.

2. The method of claim 1 wherein said expressed RNA comprises one or more polyadenylated RNA molecules.

3. The method of claim 2 wherein said cDNA molecule is prepared from a polyadenylated RNA molecule.

4. The method of claim 3 wherein said first amplicon is 5′ to the nucleotide in said cDNA molecule corresponding to the start of the polyadenylate tail of said polyadenylated RNA molecule, or said first amplicon comprises said nucleotide.

5. The method of claim 1 wherein said second amplicon contains sequences present in said first amplicon.

6. The method of claim 1 wherein the sequence of said second amplicon does not overlap with the sequence of said first amplicon.

7. The method of claim 1 wherein said amplifying is by use of quantitative PCR.

8. The method of claim 1 wherein said amplifying of the first and second amplicon is by use of quantitative PCR to produce a first Ct value for the amplification of the first amplicon and a second Ct value for the amplification of the second amplicon and said comparing is of the first and second Ct values.

9. The method of claim 9 wherein said comparing comprises determination of the difference between the first and second Ct values.

10. The method of claim 1 wherein said amplifying of the first and second amplicon is by use of quantitative PCR to produce a first Ct value for the amplification of the first amplicon and a second Ct value for the amplification of the second amplicon and
said comparing comprises determination of the amount of RNA corresponding to the first Ct value and the amount of RNA corresponding to the second Ct value followed by comparing the two RNA amounts.

11. The method of claim 10 wherein comparing the two RNA amounts comprises determining a ratio of the amount of RNA corresponding to the first amplicon and the amount of RNA corresponding to the second amplicon.

12. The method of claim 1 wherein said sample is from a human subject.

13. The method of claim 1 wherein said sample is an FFPE sample.

14. The method of claim 1 wherein said cDNA molecule encodes all or part of β-actin.

15. The method of claim 1 wherein said population of cDNA is prepared by reverse transcription using a oligo- or poly-dT primer.
